(19) United States
(12) Patent Application Publication
(10) Pub. No.: US 2026/0020984 A1
(43) Pub. Date: Jan. 22, 2026

(54) A SYSTEM FOR A TREATMENT WITH LASER OF PIGMENTED OCULAR TISSUES

(71) Applicant: Pedro GRIMALDOS RUIZ, BARCELONA (ES)

(72) Inventor: Pedro GRIMALDOS RUIZ, BARCELONA (ES)

(21) Appl. No.: 19/155,343

(22) PCT Filed: Mar. 1, 2024
(86) PCT No.: PCT/EP2024/055344

§ 371 Date: Aug. 11, 2025

(30) Foreign Application Priority Data
Mar. 2, 2023 (EP) 23382191.7

(51) Int. Cl. A61F 9/008

(52) U.S. Cl. CPC A61F 9/00802; A61F 2009/00846; A61F 2009/00851; A61F 2009/00863; A61F 2009/00868; A61F 2009/00876; A61F 2009/00882

(57) ABSTRACT

A system for a treatment with laser of pigmented ocular tissues. the system comprising a first subsystem (1) for imaging an pigmented ocular tissue of a person. a second subsystem (2) for planning a laser treatment of the pigmented ocular tissue, and a third subsystem (3) for performing the laser treatment, wherein: the first subsystem (1) comprises image analysis means (15), a camera, an optical coherence tomography apparatus (12) and at least one first head positioner (13, 14); the second subsystem (2) comprises a computer (21); the third subsystem (3) comprises an eye tracker, three or more lasers (111) of respective different wavelengths, an optical assembly (91), control means (115) and a second head positioner (92).

### CROSS-REFERENCE TO RELATED APPLICATIONS

This application is a 35 U.S.C. § 371 National Stage patent application of PCT/EP2024/055344, filed on 1 Mar. 2024, which claims the benefit of European patent application no. 23382191.7, filed on 2 Mar. 2023, the disclosures of which are incorporated herein by reference in their entirety.

### TECHNICAL FIELD

The present disclosure relates to a system for a treatment with laser of pigmented ocular tissues. A pigmented ocular tissue may be an ocular tissue that comprises melanin which is a known pigment. The treatment may be a surgical operation for medical or cosmetic purposes, using a laser beam. In particular, the operation may be directed towards one or more parts of the eye with pigmented ocular tissues, especially parts which comprise melanocytes, such as the iris, the retina or the trabeculum. The system may be an apparatus, machine or module, or may comprise a set of apparatuses, machines, modules or combinations thereof. Said system may alternatively or complementary be called system for laser eye surgery.

### BACKGROUND

There are known systems and machines for surgically operating an eye with laser. A particular type of surgical operations on the eye concerns the laser treatment of pigmented ocular tissues which comprise melanocytes, such as for example the iris, the retina and the trabeculum. Patent application document with publication number WO 2019/110855 A1 describes a system and intracellular method for depigmenting the iris using a laser, with an ultra-short duration of the trigger pulse and with a wavelength specific for melanin, which causes destruction of the intracellular melanosomes. It may be required for medical or cosmetic purposes to reduce the concentration of the melanin found in the aforementioned ocular tissues of the of eye, and this can be done using a laser beam. However, this is a particularly challenging task because the laser beam should be controlled such that the safety of the operation is improved, and such that the laser beam specifically affects the treatment with laser of pigmented ocular tissues and regions of interest containing the melanocytes and the melanin. Moreover, these types of operations often require scanning the laser beam over considerably large areas or regions of the eye, which may be a slow process, and should be done with high precision and control for safety reasons and to account for the fact that the concentration or type of melanin may vary across the scanned region. If the operation is not performed with high level of precision and control, this may negatively impact the safety and medical and/or cosmetic objective of the overall operation. The known conventional systems for the laser treatment of the eyes are not suitable for performing the aforementioned types of operations in a manner that can meet the respective medical and/or cosmetic objectives and simultaneously offer high levels of control, speed and safety.

Therefore, there is a need for a system for laser eye surgery, that overcomes the drawbacks of the conventional ones.

### SUMMARY

The present disclosure overcomes the drawbacks of conventional systems for operating an eye using lasers. In particular, the present disclosure allows for operating an eye using a laser beam, with a high level of control and precision. Moreover, the present disclosure may advantageously allow for greatly improving the speed of the treatment, such that the duration of the overall operation is greatly reduced. Also, the present disclosure allows for improving the safety of the overall operation on the eye.

In a first aspect of the disclosure, a system for a treatment with laser of pigmented ocular tissues is provided. The system comprises a first subsystem for imaging a pigmented ocular tissue of an eye of a person, a second subsystem for planning a laser treatment of the pigmented ocular tissue, and a third subsystem for performing the laser treatment.

The first subsystem comprises image analysis means, a camera, an optical coherence tomography apparatus and at least one first head positioner for positioning a head of the person such that the pigmented ocular tissue can be imaged by the camera and the optical coherence tomography apparatus. The camera is configured to capture at least one color image of the eye and the pigmented ocular tissue. The optical coherence tomography apparatus is configured to capture at least one optical coherence tomography image of the pigmented ocular tissue. Preferably, the optical coherence tomography apparatus is configured to capture at least one optical coherence tomography image of the eye and the pigmented ocular tissue. The image analysis means is configured to process the at least one color image and the at least one optical coherence tomography image for performing a set of measurements. The measurements comprise a biometry of the eye, a densitometry, a pachymetry, and a colorimetry of the pigmented ocular tissue. The colorimetry comprises mapping or identifying the color of different regions of the pigmented ocular tissue. The image analysis means is further configured to produce data related to the set of measurements.

The second subsystem comprises a computer which is configured to process the data related to the set of measurements. The computer is also configured to record a set of instructions. The set of instructions comprises values of laser parameters to be set for a laser scan across different regions of the pigmented ocular tissue. The set of instructions also comprises an indication of one or more lasers or laser wavelengths to be used during the laser scan for each one of the different regions of the pigmented ocular tissue according to the color of the different regions.

The third subsystem comprises an eye tracker, three or more lasers of respective different wavelengths, an optical assembly, control means configured to control the optical assembly and the three or more lasers for executing the laser scan, and an additional head positioner for positioning the head of the person such that the pigmented ocular tissue can be treated with a laser beam produced by any of the three or more lasers. The optical assembly is configured to scan the laser beam across the different regions of the pigmented ocular tissue. The eye tracker is communicatively connected to the control means and is configured to track movements of the eye during the laser scan, and to provide to the control means tracking information related to the tracked movements. The control means is configured to: receive and process the set of instructions and the tracking information; control the optical assembly according to the tracking information and the set of instructions; select, among the three or more lasers and according to the set of instructions, the laser that produces the laser beam for each one of the different regions during the laser scan; and set the values of the laser parameters during the laser scan across the different regions.

The system according to the first aspect of the disclosure, can also be an apparatus. The first, second and third subsystems of the system, may also be respective modules of the system or apparatus. The first system may also be called imaging station. The second system may also be called planner. The third subsystem may also be called laser station.

The first subsystem allows for imaging the pigmented ocular tissue of the person or patient on which the laser eye treatment or treatment is to be performed. The plurality of the measurements that can be taken by means of the first subsystem allows for a correct and qualitative characterization and evaluation of the eye prior to performing the laser surgery. Specifically, the colorimetry permits identifying the type of the melanin on the region of the eye on which the laser beam is to be applied. This, considering that different melanin types have different optical absorption properties (e.g. optical absorption spectra) from each other, in turn permits choosing correctly the wavelengths of the laser beam, and therefore, choosing the laser to be used for generating said laser beam. Moreover, the color of different regions of the eye can be considered as an indication of the concentration of the melanin in these regions.

Also, mapping or identifying the color of the different regions of the eye before the application of the laser is a particularly important for certain types of laser surgery, such for example laser surgery, e.g. cosmetic iridoplasty, for altering the color of the iris. In the later case, the color of the different regions of the iris before the laser operation, together with the iris color that is sought to be obtained with the laser surgery, should be taken into account for adjusting the laser parameters, e.g. the laser wavelength, used in the different regions of the iris during the operation.

The densitometry, i.e. measuring/estimating directly or indirectly the density of the light-sensitive matter on regions of the eye, may allow for adjusting the energy of the laser beam to be applied on said regions. Hence, regions of lower optical density may require the application of lower laser energy compared to regions of higher optical density, such that the laser beam primarily or exclusively affects and the pigmented ocular tissue's surface that is exposed to the laser beam, without the laser beam travelling and reaching at significant depths/layers below said surface. This may be a particular important task to achieve in certain types of operations for which the goal is to reduce the concentration of melanin located on the surface of the pigmented ocular tissue, without damaging the tissue that is located below the melanin-containing surface. The density of said regions may be estimated from the at least one image of the pigmented ocular tissue. In a preferred embodiment of the disclosure, the densitometry comprises mapping or identifying the density of the different regions of the pigmented ocular tissue. Also, in a preferred embodiment of the disclosure, the laser parameters comprise an energy (laser energy) of the laser beam; the densitometry comprises mapping or identifying the density of the different regions of the pigmented ocular tissue; the computer of the second subsystem is configured to record values of the energy to be used during the laser scan for each one of the different regions according to the density of the different regions; and preferably the computer of the second subsystem is configured to automatically generate the values of the energy as a function of the density of the different regions.

The pachymetry i.e. the measurement or estimation of the thickness of the pigmented ocular tissue, can be done in different possible ways. The optical coherence tomography (OCT) can be used for directly measuring said thickness and how the thickness changes across the different regions of the pigmented ocular tissue. However, it may be also possible to get an indirect estimation of said thickness from the image obtained from the camera. The information related to the thickness of the pigmented ocular tissue can advantageously allow for adjusting and optimizing the duration (width) of the laser pulse, so that the laser pulse primarily interacts with the aforementioned surface of the pigmented ocular tissue, in order to prevent the laser beam reaching and damaging the tissue layers further below said surface. It is noted that in a preferred embodiment according to the disclosure, the pachymetry comprises mapping or identifying the thickness of the different regions of the pigmented ocular tissue. Also, in a preferred embodiment of the disclosure, the laser parameters comprise a pulse duration of the laser beam; the pachymetry comprises mapping or identifying the thickness of the different regions of the pigmented ocular tissue; the computer of the second subsystem is configured to record values of the pulse to be used during the laser scan for each one of the different regions according to the thickness of the different regions; and preferably the computer of the second subsystem is configured to automatically generate the values of the pulse duration according (as a function of) the thickness of the different regions.

The biometry, i.e. the analysis of the dimensions of the eye and/or of the area of the region of interest of the pigmented ocular tissue, advantageously may allow for adjusting the laser frequency during the laser treatment of said region, so that the laser treatment is fast i.e. it has a small duration. When the laser beam must be scanned over a large area, the laser frequency may be chosen to be larger compared to the laser frequency used for scanning a smaller area, so that scanning and correspondingly treating with the laser the large area does not take (much) longer compared to scanning and treating the smaller area. Hence, in a preferred embodiment of the disclosure, the laser parameters comprise a laser pulse frequency; the biometry comprises identifying one more dimensions of the eye; the computer of the second subsystem is configured to record values of the laser pulse frequency to be used during the laser scan according to (as a function of) the one or more dimensions of the eye; and preferably, the computer of the second subsystem is configured to automatically generate the values of the laser pulse frequency as a function of the one or more dimensions. In preferred embodiment, the laser frequency is of between 100-300 Hz.

In a preferred embodiment of the disclosure the set of measurements comprise a topography of the pigmented ocular tissue, particularly a topography of the different regions of the pigmented ocular tissue. The topography can advantageously allow optimizing the diameter of the laser beam used during the laser scanning and laser treatment of the pigmented ocular tissue, because if the tissue on the surface of which the laser beam acts comprises fine topographical surface features such as recessions and elevations, then it may be required that that the diameter (spot size) of the laser beam is comparable to, or smaller than, the dimensions of said topographical features. This can in return advantageously permit that the energy of the laser beam/pulses can be sufficiently delivered on said fine features for causing thereabouts the desired effect. Hence, in a preferred embodiment the laser parameters comprise a diameter of the laser beam; the set of measurements comprise a topography of the different regions of the pigmented ocular tissue; the computer of the second subsystem is configured to record values of the diameter to be used during the laser scan for each one of the different regions according to the topography of the different regions; and preferably, the computer of the second subsystem is configured to automatically generate the values of the diameter according to (i.e. as a function of) the topography of the different regions. More preferably, said topography comprises mapping or identifying a surface morphology of the different regions of the pigmented ocular tissue, and the computer of the second subsystem is configured to automatically generate the values of the diameter according to (i.e. as a function of) the surface morphology of the different regions

The image analysis means may be or comprise a first computer that is suitable, i.e. configured, for processing images. This first computer may have a graphics cart, but alternatively or complementary the computer may have a graphics processing unit (GPU) built in a central processing unit (CPU) of the computer. The image analysis means may be located physically close to the camera, the OCT apparatus and other parts of the first subsystem, or may be located in a remote location and be connected to the rest of the first subsystem. For example, the image analysis means may be or comprise a computer server that is connected to the rest of the system via the internet or a local network. The data related to the set of measurements may include the different thicknesses, the different colors or color tones and the different densities of the different regions of the pigmented ocular tissue being imaged by the OCT system and the camera. Alternatively or complementary, said data may include one or more graphs showing the different thicknesses, colors and/or densities.

Preferably, the processing of the at least one color image is done, at least in part, automatically by the image analysis means for thereby automatically performing any or all of the set of measurements. Advantageously, the automatic performance of any or all of the set of measurements may contribute to the reduction of the time required for the overall evaluation and treatment of the eye of the patient using the system of the disclosure, and may also advantageously enable the error-free execution of the characterization of the eye on the basis of algorithms and protocols on which said automatic image processing and related measurements may be based. In particular, the automatic performance of the colorimetry can advantageously offer accurate information regarding the color map of the pigmented ocular tissue so that the wavelength of the laser may be set accurately and correctly according to the color or color tone of each region of the eye. Therefore, in a preferred embodiment of the disclosure the image analysis means is configured to automatically perform the set of measurements, or any of the biometry, the densitometry, the topography, the pachymetry, and the colorimetry, preferably the colorimetry.

The second subsystem may be called “planner” because it may allow for planning in detail the laser operation based on the measurements and related data produced with the first subsystem. For this purpose, the computer of the second subsystem is configured, e.g. comprises suitable software, for processing the data related to the set of measurements. The computer of the second subsystem may also be, comprise or act as the image analysis means of the first subsystem, or may be connected, e.g. via a local area network or the internet, to the image analysis means. Hence, if the image analysis means comprise a first computer, the computer of the second subsystem may be called second computer so that it can be distinguished from the first computer. Hence, there is also contemplated the optional case that the transfer of data from the first to the second computer can be realized by means of a computer readable medium on which the data are stored at the first computer so that when the computer readable medium is connected and read by the second computer the latter can access said data. Similarly, a computer readable medium may be used for transferring data or instructions from the computer of the second subsystem to the third subsystem.

Importantly, the computer of the second subsystem allows to record the set of instructions, and said recording may be made automatically or can be made manually by a user of the computer. In a preferred embodiment of the disclosure, the computer of the second subsystem is configured to generate automatically at least partially the set of instructions. The automatic generation of the set of instructions may advantageously contribute to an overall reduction of the time required for planning the laser operation, and may advantageously also relieve the user of the system from trivial or repetitive work which could be done automatically and free of errors on the basis of pre-determined specific algorithms and protocols. This way, advantageously the user of the system may have more time for inspecting and if required correcting or further optimizing the planned operation. Hence, it can be understood that preferably the computer of the second subsystem is further configured to enable a manual input or modification by a user of anyone instruction of the set of instructions. The user may be an ophthalmologist or eye surgeon who evaluates the data related to the set of measurements and chooses accordingly the laser parameters, e.g. the one more laser wavelengths, to be used during the laser operation.

Preferably the set of instructions may also include instructions for implementing a scanning path during the laser scan which can be executed by means of the third subsystem, or said set of instructions may include a routine which defines said scanning path. Therefore, in a preferred embodiment of the disclosure the set of instructions comprises a routine which defines a scanning path to be followed by the laser beam during the laser scan. In a preferred embodiment, the scanning path is a flying-spot scanning path. The “flying-spot” type of scanning, can be understood as being a scanning during which every two consecutive laser pulses are fired on the eye at distant spots/places with respect to each other, so that two regions being treated by two consecutive pulses do not overlap or border each other. This advantageously may decrease the danger of thermal damage on the eye and, hence, may further improve the safety of the procedure. Said “flying-spot” scanning path or mode of operation may be included in the set of instructions being recorded with the planner, or may be generated or enabled by the third subsystem when or before the latter executes the laser scan. It is noted that preferably the computer of the second subsystem is configured to automatically generate the aforementioned routine, and/or the control means are configured to control the optical assembly according to the routine. Therefore, in a preferred embodiment, the planner, i.e. the second subsystem, enables the automatic or manual determination of the scanning path to be followed using the optical assembly of the third subsystem. For this purpose, most preferably the optical assembly comprises a galvo scanner, or other similar component, for scanning the laser beam, e.g. for scanning the beam along said scanning path. In the latter embodiment, the control means of the third subsystem may, using a feedback from the eye tracker and the set of instructions, control the optical assembly and/or a galvo scanner in said assembly, so that the scanning path is followed during the scanning across the different regions of the pigmented ocular tissue being treated with the laser. Likewise, as mentioned further above, the control means is also configured to set the values of the laser parameters during the laser scan across the different regions of the pigmented ocular tissue.

Said laser parameters may include for example a first laser wavelength to be used on a first region of the pigmented ocular tissue, and a second wavelength to be used on a second region of the pigmented ocular tissue when treating the first and the second region with the third subsystem. Alternatively or complementary, the specified laser parameters may not comprise a direct indication of the laser wavelength, but instead may comprise an indication of the laser, e.g. the laser model or type, to be used. The three or more lasers of the third subsystem are of respective different wavelengths, i.e. each of said three or more lasers is configured to generate a laser beam of a different wavelength compared to the other lasers. Hence, an indication of the laser to be used among the three or more lasers may act as an indication of the laser wavelength to be used. However, any of the three or more lasers may be configured to controllably produce more than one laser wavelengths, in which case the parameters to be specified by means of the planner (the second subsystem) may include an indication of the laser wavelength or of an operation mode of the laser that can result to the laser producing said laser wavelength. It is noted that the third subsystem may preferably comprise more than three lasers of respective different wavelengths, and for example may comprises four lasers.

The laser parameters the values of which can be recorded with the computer of the second subsystem, may include some parameters which are mentioned further below, namely the energy, the pulse duration and the frequency. The laser parameters may further include the wavelength and the spot size of the laser beam. In a preferred embodiment the laser parameters include (comprise) the energy, the pulse duration, the frequency, the wavelength and the spot size of the laser beam. The instructions comprising said values and being recorder with the second subsystem preferably have the form of a computer code or file which can be accessed and processed by the control means. The control means may be or comprise an appropriately configured electronic card, unit and/or computer, e.g. a third computer. There are known electronic cards and computer systems which are connectable with lasers, galvo scanners and eye trackers for controlling the lasers and the galvo scanners during an eye surgery that involves a laser scan. The set of instructions recorded by the second subsystem and received and processed by the control means, advantageously allows for improving the safety of the laser treatment and for optimizing the laser scan so that the latter has the desired effect on the eye. The eye tracker and the tracking information provided by the eye tracker, may advantageously allow for avoiding mistargeting the laser due to possible unplanned movement of the eye during the laser scan. Hence, the eye tracker allows for using the laser parameters recorded by the second subsystem on the correct regions of the pigmented ocular tissue for which said parameters were recorded and intended to be used. Likewise, the eye tracker may allow for avoiding applying the laser beam on wrong parts of the eye which can be damaged if the laser beam acts on said wrong parts of the eye. In a preferred embodiment the eye tracker is a seven-dimensional eye tracker. Advantageously the use of a seven-dimensional eye tracker may improve the safety of the system.

As mentioned, advantageously the system according to the disclosure is particularly suitable to be used for reducing the amount of melanin found in different parts of the eye such as the iris, the trabeculum or the retina. The melanin is produced in melanocytes of the eye. Therefore, in preferred embodiments of the system, the pigmented ocular tissue that is treated using the system comprises the iris, the trabeculum, the retina and/or or any eye tissue that contains melanocytes.

The application of the laser on the pigmented ocular tissue may result to the ablation or the apoptosis of the melanocytes which are located on the pigmented ocular tissue that is to be treated with the laser. Typically, the laser parameters may be set for selecting between two modes of operation, the first mode being for causing the ablation of the melanocytes, and the second mode being for causing the apoptosis of the melanocytes. Hence, in a preferred embodiment of the disclosure, the wavelengths of the three or more lasers and/or the recorded values of the laser parameters are such that the laser beam produces, during the laser scan, an ablation or an apoptosis of the melanocytes located on the pigmented ocular tissue. Preferably the wavelengths of the three or more lasers, and/or the recorded values of the laser parameters are such that the laser beam is non-ablative and produces an apoptosis of the melanocytes during the laser scan. Typically, two parameters that significantly affect the type of the effect the laser has on the treated pigmented ocular tissue, are the power density and pulse duration of the laser. Typically, a laser which has a wavelength of approximately 532 nm or 561 nm or 577 nm, a power density of about 10⁹ W/cm² and a pulse duration of between 1 ns and 40 ns, will have a photo-ablative effect on the melanin containing pigmented ocular tissue, and can cause the ablation of the melanocytes. For causing the apoptosis instead of the ablation, typically the power density should be set to lower values and the pulse duration should be set to higher values, for example the power density may be in the order of about 10⁶ W/cm² and the pulse duration may be in the order of about 1 ms, e.g. the pulse duration may have a value of be between 0.1 ms and 3 ms. It is noted that the ablation of the melanocytes may be accompanied be the increase of the pressure at the treated region of the eye, and for this reason the first mode operation may require that the treatment involves frequent pauses or treatment sessions so that pressure does reach or exceed dangerous levels. This may not be required with the second mode operation. Hence, advantageously, the second mode operation, i.e. choosing/setting the laser parameters for causing an apoptosis of the melanocytes, can be significantly faster compared to the first or other modes of operation.

The first, the second and the third subsystems may be, i.e. have the form of, separate respective modules or apparatuses, or preferably may be integrated (e.g. be in physical contact) with each other forming a single apparatus. If the system according to the disclosure has the form of a single apparatus, then advantageously the space occupied by the system can be minimized and the users of the system, said users typically being ophthalmologist or eye surgeons, benefit from not having to move and/or transfer data from one subsystem to the next one. However, it is noted that even when the first, second and third subsystems of the system are not integrated with each other, they may still be connectable to each other, via wire or wirelessly, for enabling the ease transmission of data between them. An advantage related to the subsystems being distinct separate modules, is that they may separately fit in respective spaces in which a single big apparatus would not fit.

In a preferred embodiment of the disclosure, the third subsystem further comprises a flare meter which is configured to detect the emission of particulates from the eye or the pigmented ocular tissue. The detection of particulates emitted by the pigmented ocular tissue during the laser treatment advantageously greatly improves the safety of the system because such particles, especially at high concentrations, can indicate the occurrence of unwanted and potentially dangerous for the patient laser-induced sublimation processes and/or the increase of ocular pressure. Preferably the flare meter is a digital flare meter. The use of a digital flaremeter (flare meter) in a laser surgery (sub) system can advantageously facilitate the compact integration of the flaremeter with the rest of the system's components, so that the overall third subsystem is compact. The digital flaremeter preferably may comprises a computer, e.g. a third computer, of the third subsystem, and may also comprise a digital camera connected to said third subsystem's computer, the latter being configured to process images of the eye captured by the digital camera for detecting therein particles being emitted by the pigmented ocular tissue. For this purpose, the third subsystem, and in particular the optical assembly, may comprise a microscope optically coupled to the said digital camera so that the camera is focused on the eye, and preferably is focused at the dark/black pupil of the eye. The images captured and processed by the digital camera are or comprise preferably black/white or grayscale images so that advantageously there is a significant contrast between any particles being emitted and the background which originate from said dark/black pupil. The computer processing said images may preferably have software which when executed can automatically detect said particles in the images. Advantageously, the aforementioned (preferable) options regarding the flaremeter, may offer an automated detection of emitted particulates, for further improving the safety of the system. In a preferred embodiment wherein the third subsystem comprises a flare meter, the latter upon detecting the emission of particulates during the laser scan is configured to trigger the control means to stop the laser scan. As mentioned, the control means may be or comprise a computer, e.g. the aforementioned third computer, which can be the same computer as the one mentioned further regarding a possible configuration of the digital flaremeter. Hence, a computer program related to the operation of said digital flaremeter may also comprise instructions for stopping the laser scan upon the detection of particulates. This way advantageously the safety of the system can be further improved.

A second aspect of the disclosure concerns the aforementioned flare meter for detecting the emission of particulates from an eye or a pigmented ocular tissue, wherein the flare meter comprises a computer, and also comprises a camera for capturing images of the eye, and wherein the camera is connected to the computer; and the computer is configured to process images of the eye captured by the camera for detecting therein (i.e. in the images) particulates being emitted by the eye or the pigmented ocular tissue. Preferably the camera is focused on or within the pupil of the eye.

Preferably the camera of the flare meter is a digital camera. More preferably the flare meter of the second aspect of the disclosure is a digital flaremeter. The camera may preferably be configured and/or configurable for capturing said images of the eye.

In a preferred embodiment, the computer of the digital flare meter, for the purpose of processing the images for detecting within said images particulates (particles) being emitted by the eye or the pigmented ocular tissue, utilizes an image analysis program (i.e. software for image analysis) which involves the use of artificial intelligence. Hence, preferably the computer of the flare meter is programmed to execute one or more algorithms of artificial intelligence for analyzing the images and detecting particulates in said images. In a non-limiting example, the aforementioned image analysis may involve/utilize/use one or more pretrained artificial intelligence models to detect or analyze one or more visible features, e.g. particulates, from the analyzed images.

The flare meter of the second aspect of the disclosure differs from conventional flare meters which are based on the amplification of a signal generated by a laser or an LED (light emitted diode). Said conventional flare meters are generally bulky and stand-alone instruments which cannot be integrated and used with eye laser surgery systems. However, in contrast to said conventional flare meters, the flare meter of the second aspect of the present disclosure advantageously may be used as a standalone system/instrument, or alternatively may also be integrated with an apparatus (system) for ocular diagnosis (i.e. eye examination) or an apparatus for eye laser treatment. Hence, in contrast to the conventional flare meters, the flare meter of the second aspect of the disclosure may advantageously be used for monitoring an eye during a laser treatment of the eye. Hence, advantageously the flare meter of the second aspect of the disclosure offers versatility, and when is optionally incorporated in a system (apparatus) for ocular diagnosis or eye laser treatment, allows for completing and improving the function and safety of said system.

In a preferred embodiment, the images captured and processed by the digital camera are or comprise black/white or grayscale images. This may advantageously allow for having within the images a significant contrast between any particles being emitted and the background.

Preferably, said background originates from the eye's pupil, as mentioned further above.

In a preferred embodiment, the computer processing said images may preferably have software which when executed can automatically detect said particles in the images.

In a preferred embodiment, the flare meter further comprises an LED light for illuminating the eye. Illuminating the eye may with the LED light may advantageously facilitate improving the quality of the images captured by the camera. More preferably, said LED light is substantially white.

A third aspect of the disclosure concerns an apparatus for eye examination or eye treatment, wherein said apparatus comprises the flare meter of the second aspect of the disclosure. Said apparatus may preferably be a system according to the first aspect of the disclosure, or it may be a different type of system. In a preferred embodiment of the third aspect of the disclosure, the apparatus is for eye treatment, wherein said treatment is a laser treatment (i.e. treatment by means of laser) of the eye, more preferably said laser treatment being laser surgery.

Preferably, the apparatus of the third aspect of the disclosure, also comprises a microscope optically coupled to the camera so that the camera is focused on the eye, and preferably is focused at the dark/black pupil of the eye.

In a preferred embodiment of the third aspect of the disclosure, wherein said apparatus is for eye treatment and comprises a laser for the treatment of the eye, said laser comprising an interlock, the flare meter of the apparatus upon detecting particulates being emitted by the eye or the eye's pigmented ocular tissue, or upon detecting that a concentration of said particulates exceeds a predetermined value, is configured to activate the interlock of the laser for stopping the function of said laser. This may advantageously allow for avoiding or reducing the potential danger that may arise if one or more parameters of the laser surpass(es) any respective safety limit(s). It is noted that in the optional case that the system of the third aspect of the disclosure comprises a laser which comprises an interlock, and the system also comprises control means for controlling said laser, then said interlock may also be considered as part of said control means. Likewise, in a preferred embodiment of a system according to the third aspect of the disclosure, wherein said system is for laser eye treatment, the flare meter may advantageously allow for testing the safety of the system's laser before the actual treatment (operation) takes place, and/or may advantageously allow for evaluating the evolution of the laser treatment during said treatment. Testing the safety of the laser to be used for a particular eye treatment may be particularly useful in the optional cases that for said eye treatment the parameters of the laser should be correctly adjusted or that the exact laser to be used should be correctly selected. Advantageously, in the latter optional cases, the use of the flare meter of the second aspect of the disclosure may allow for correctly adjusting said laser parameters or for correctly selecting the laser to be used for a particular eye treatment.

Additional advantages and features of the disclosure will become apparent from the detailed description that follows and will be particularly pointed out in the appended claims.

### BRIEF DESCRIPTION OF THE DRAWINGS

To complete the description and in order to provide for a better understanding of the disclosure, a set of drawings is provided. Said drawings form an integral part of the description and illustrate an embodiment of the disclosure, which should not be interpreted as restricting the scope of the disclosure, but just as an example of how the disclosure can be carried out. The drawings comprise the following figures:

FIG. 1 illustrates an embodiment of the system according to the disclosure.

FIG. 2 illustrates an embodiment of the system according to the disclosure.

FIG. 3 illustrates a first subsystem of an embodiment according to the disclosure.

FIG. 4 illustrates data related to a set of measurements performed with a first subsystem of an embodiment according to the disclosure.

FIG. 5 illustrates data related to a topography performed with a first subsystem of an embodiment according to the disclosure.

5 FIG. 6 illustrates an optical coherence tomography image and related data acquired with a first subsystem of an embodiment according to the disclosure.

FIG. 7 illustrates a computer of a second subsystem of an embodiment according to the disclosure.

FIG. 8 illustrates information displayed on the computer of FIG. 7.

FIG. 9 illustrates a third subsystem of an embodiment according to the disclosure.

FIG. 10 illustrates the positioning of a patient at a third subsystem of an embodiment according to the disclosure, for the treatment with the laser.

FIG. 11 illustrates schematically certain components of a third subsystem of an embodiment according to the disclosure, for performing an laser scan on an eye.

FIG. 12 illustrates an image of an eye taken with the third subsystem during the laser treatment.

FIG. 13 illustrates an image of an pigmented ocular tissue, the image taken with the third subsystem of an embodiment which comprises a digital flaremeter.

FIG. 14 illustrates types of eye movement being tracked with an eye tracker of an embodiment of the disclosure, during the laser scanning.

FIG. 15 illustrates the application of a laser beam on the eye, for laser trabeculoplasty for glaucoma.

FIG. 16 illustrates the application of a laser beam on the eye, for retinal coagulation by laser for retinal diseases.

### DETAILED DESCRIPTION OF THE DRAWINGS

The following description is not to be taken in a limiting sense but is given solely for the purpose of describing the broad principles of the disclosure. Next embodiments of the disclosure will be described by way of example, with reference to the above-mentioned drawings, showing apparatuses and results according to the disclosure.

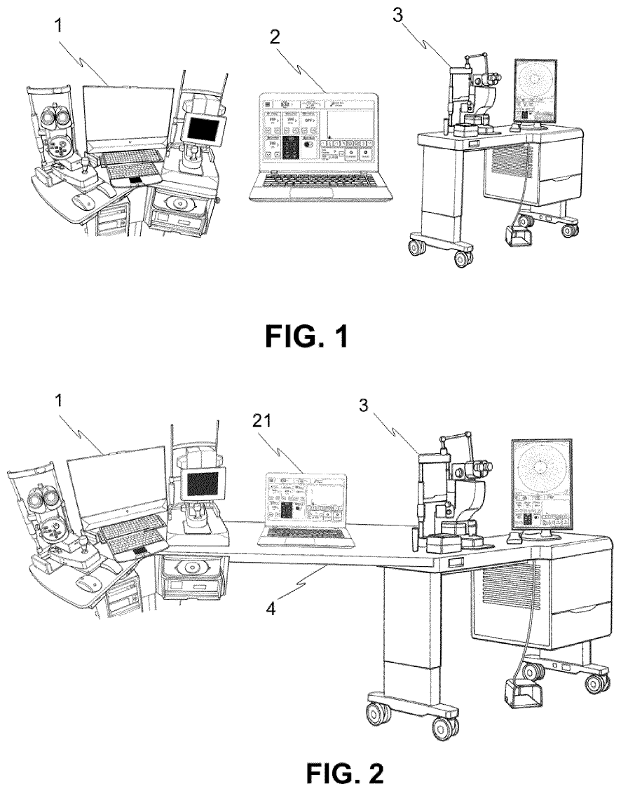

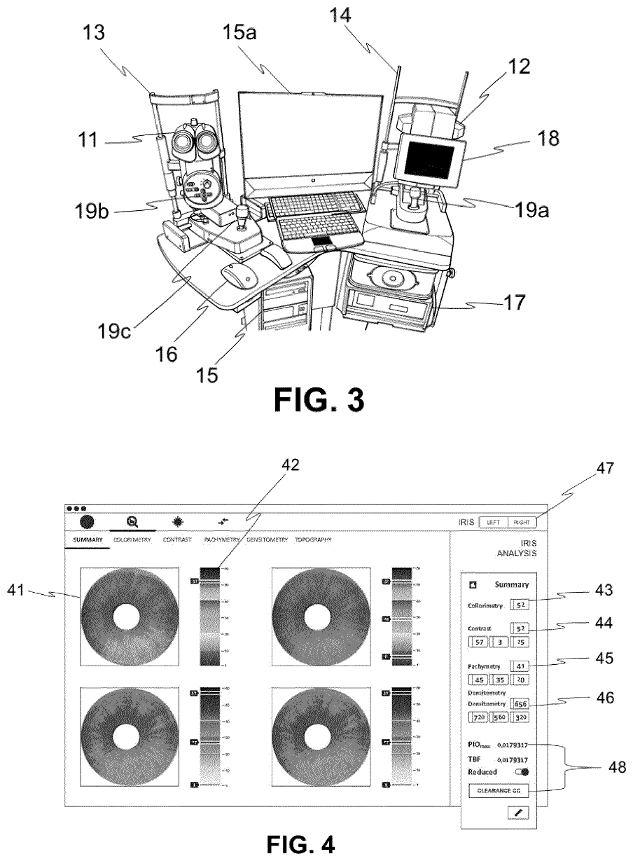

Embodiments of a system for a treatment with laser of pigmented ocular tissues according to the disclosure are explained next with reference to FIG. 1-14. FIG. 1 and FIG. 2 show two respective embodiments of a system according to the disclosure, each embodiment comprising a first subsystem 1 for imaging a pigmented ocular tissue of a person, a second subsystem 2 for planning a laser treatment of the pigmented ocular tissue, and a third subsystem 3 for performing the laser treatment. The embodiment of FIG. 1 is modular meaning that each of the three subsystems 1, 2, 3 is a respective separate module that may operate autonomously. In the embodiment of FIG. 2 the three subsystems are integrated with each other forming a single workstation that further comprises a bench 4 which extends from the first to the third subsystem, and the computer 21 of the second subsystem is located on said bench in between the other two subsystems. The first subsystem shown in FIG. 1-3 comprises an ophthalmic microscope 11 with a camera, an optical coherence tomography (OCT) apparatus 12, two first head positioners 13, 14 for respectively positioning the head of the patient in front of the ophthalmic microscope 11 and the OCT apparatus 12 so that the eye can be imaged using respectively the camera and the OCT apparatus 13. It is noted that there is contemplated the option of the first subsystem having a single first head positioner for positioning the head of the patient in front of the microscope 11 and the OCT apparatus 12. The camera of the shown ophthalmic microscope 11 is configured to capture at least one color image of the eye (the pigmented ocular tissue), and the OCT apparatus 12 is configured to capture at least one optical coherence tomography image of the pigmented ocular tissue and the eye. The shown OCT apparatus 12 of FIG. 3 comprises a control unit 17 connected to a first joystick 19a that permits a manual selection of the region of the eye to be imaged. The shown OCT apparatus 13 of FIG. 3 further comprises a screen 18. The ophthalmic microscope 11 shown in FIG. 3 further comprises control keys 19b for controlling the microscope 11, and a second joystick 19c that permits the user focusing on different regions of the eye with the camera of the microscope 11. The first subsystem 1 of FIG. 3 further comprises image analysis means 15 which is a computer configured to process the at least one color image and the at least one optical coherence tomography image for performing a set of measurements which comprise a biometry of the eye, and a densitometry, a pachymetry and a colorimetry of the eye (i.e. of the pigmented ocular tissue). Said computer 15 of the first subsystem of FIG. 3 further comprises a monitor 15a for displaying said images and/or related measurements and data, and also comprises a keyboard and a mouse 16 so that the user can interact and provide an input when necessary for the measurements.

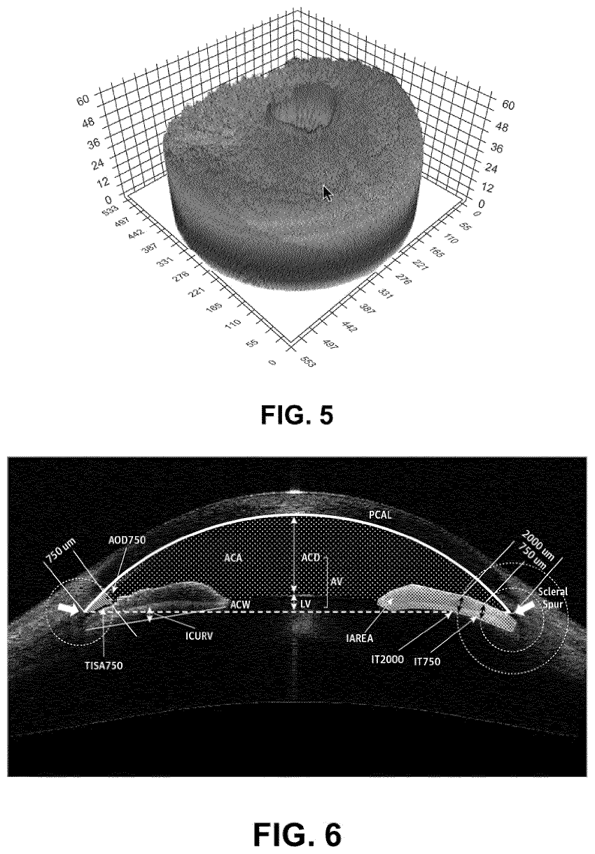

The image analysis means 15 of the first subsystem shown in FIG. 3 is further configured to produce data related to the set of measurements, and FIG. 4 show some of said data produced in a preferred embodiment that is particularly suitable for performing photoablative or non-photoablative cosmetic iridoplasty. As shown in FIG. 4, said data comprise four color images 41 (color maps) of the iris for respective different color tones (two brown tones, one blue tone, and one green tone) and also comprise values on corresponding color scales 42 related to the tone of each point or region of the iris. Various types of color scales can be used for this purpose, and in a preferred embodiment there is used a scale from 0 to 60 (i.e the maximum value in the scale is 60 and the minimum is 0). The data shown in FIG. 4 further comprise colorimetry summary data for each region/point of the iris, said summary data comprising a contrast value 44 which is the average of the two brown tone values, as well as the blue tone value and the green tone value of each point or region of the iris. There are also shown in FIG. 4 thickness values 45 related to a pachymetry performed on the iris, and density values 46 related to a densitometry performed on the iris. Moreover, the data shown in FIG. 4 further comprise some values 48 of variables which may be particularly important to know for advantageously further improving the safety of the subsequent laser operation, said variables being the trabecular blocking factor (FBT), the clearance curve (cc) and the predicted maximum ocular pressure (PIOₘₐₓ) that would be reached if the iris surface is completely removed (36.65 mmHg). Advantageously the safety and overall quality of the overall procedure, can be improved if the set of measurements performed with the first subsystem further comprise a three dimensional (3D) topography of the pigmented ocular tissue, and FIG. 5 shows an example of such measurement wherein the thickness of an imaged iris is plotted as a function of the coordinates on an x-y plane.

FIG. 6 shows an OCT image and related dimensions of the eye, said dimensions being measured as part of the biometry made with the first subsystem of a preferred embodiment of the disclosure. The image of FIG. 6 shows a cross section of a patient's eye, and therein the are marked the following: posterior corneal arc length (PCAL), anterior chamber depth (ACD), anterior chamber width (ACW), anterior chamber area (ACA), sclerar spur, lens vault (LV), anterior vault (AV), iris curvature (ICURV), iris area (IAREA), iris thickness at 750 um from the scleral spur (IT750), iris thickness at 2000 μm from the scleral spur (IT2000), iris space area at 750 μm from the scleral spur (TISA750), and angle opening distance at 750 μm from the scleral spur (AOD750).

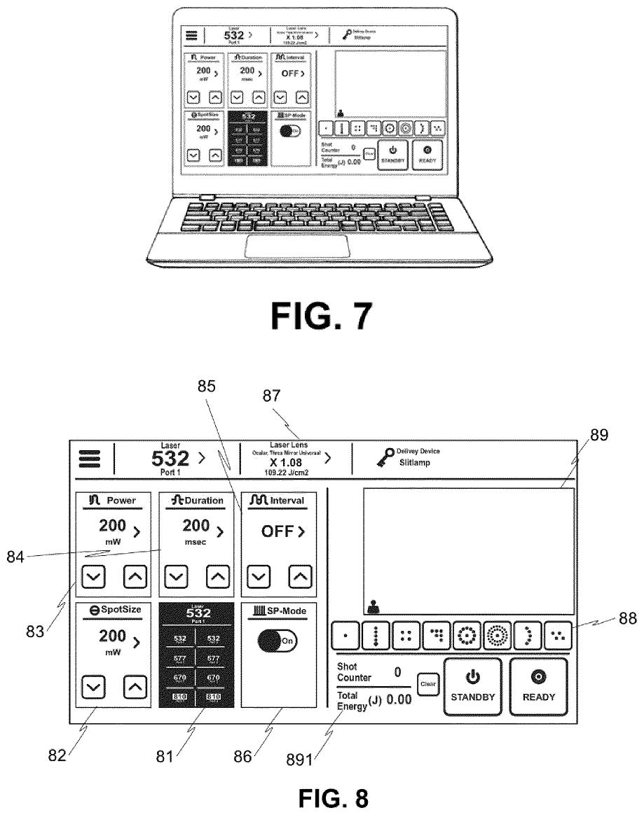

The computer 21 of the second subsystem of the embodiments of FIG. 1 and FIG. 2 is a laptop and is further shown in FIG. 7. A computer program being executed with the computer of FIG. 7 allows for processing the data related to the set of measurements, and for recording a set of instructions. Alternatively or complementary, the computer may have more than one computer programs which when executed enable processing the data from the set of measurements, and recording the set of instructions. FIG. 8 shows a screenshot from the computer program being run on the computer 21 of FIG. 7. FIG. 8 shows a graphical interface for recording values of laser parameters to be set for a laser scan across different regions of the pigmented ocular tissue. Specifically, the interface shown in FIG. 8 comprises a first section 81 with clickable features for setting the wavelength of the laser beam to be used, a second section 82 for setting the spot size (diameter) of the laser beam, a third section 83 for setting the laser power, a fourth section 84 for setting the duration of the pulse, a fifth section 85 for setting an interval between laser pulses, a sixth section 86 for selecting whether to operate the laser at a short-pulse (SP) mode, a seventh section 87 for setting a lens to be used with the laser, an eighth section 88 for setting a scanning/marking pattern, a ninth section 89 for optionally showing therein a picture or image of the pigmented ocular tissue, and additional sections 891 for showing information such as the number of shots and the estimated total energy to be applied with the laser on a treated area of the eye. It is noted that selecting the aforementioned short-pulse mode may involve adjusting/setting the duty cycle of the laser. Some laser wavelengths which are preferably used are 532 nm, 577 nm, 670 nm and 810 nm. Therefore, preferably the lasers of the third subsystem suitable for producing the aforementioned wavelengths. The value of laser energy, particularly for non-ablative cosmetic iridoplasty, may preferably set to be in the order of 1-500 μJ. The duration may preferably be of between 1 and 500 milliseconds, and the frequency may be in the order of 100-300 Hz. Hence, the lasers of the third subsystems may be configured to give a laser beam having the aforementioned pulse duration and power. The computer of the second subsystem, may have stored therein a computer program (computer code) which when executed produces automatically values for some or all of the laser parameters to be used during the laser scan. For example, the computer may be configured to: select the wavelengths to be used for each region of the pigmented ocular tissue, according to the color tone of said region; calculate the value of the duration as a function of the thickness of the region; and, calculate the laser power as a function of the density of the region. In a non-limiting example, the value of the pulse duration is set to increase as the value of the thickness increases, and the value of the laser power is set to increase as the density increases. Moreover, in a non-limiting example, the value of the laser frequency is set to increase as an estimated surface area of the scanned region of the eye-part increases, so that the scanning and overall laser treatment is effective and fast.

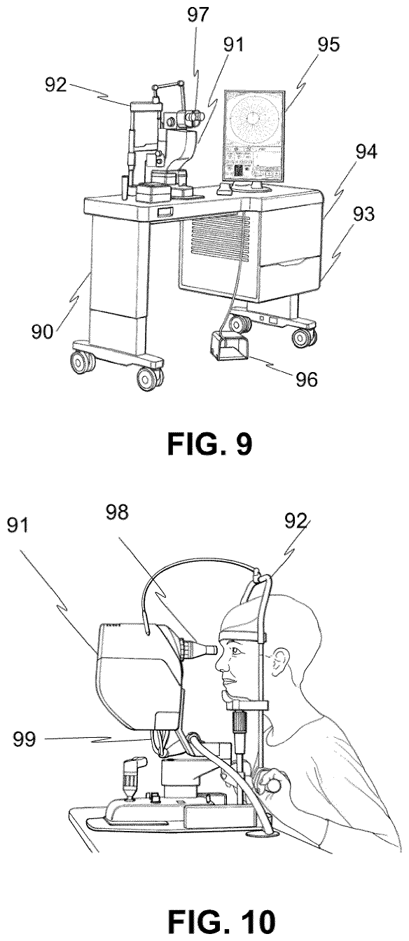

FIG. 9 shows the third subsystem of a preferred embodiment of the disclosure. The third subsystem, i.e. third module, shown in FIG. 9 comprises a movable workbench 90 on which there is the optical assembly 91 of the third subsystem, a head positioner 92 (i.e. the second head positioner 92 of the overall system) a computer within a first cupboard 93 of the workbench 90, three lasers located in a second cupboard 94 of the workbench, a display 95 connected to the computer of the third subsystem, and a foot pedal 96 which the user may use for manually firing/shooting the laser beam towards the eye once the eye is being tracked with the eye tracker and the instructions from the planner have been processed by the control means or computer of the third subsystem. The optical assembly 91 shown in FIG. 9 comprise a respective ophthalmic microscope 97 for viewing the patient's eye. Preferably, said computer of the third subsystem of FIG. 9 may be configured (e.g. have appropriate software) to enable the user of the third subsystem to view on the display 95 an image of the pigmented ocular tissue, a part or all of the set of instructions being recorded with the second subsystem, and if necessary the data related to the measurements taken with the first subsystem. Also, said computer may be or act as the control means of the third subsystem.

FIG. 10 show a part of a third subsystem which is similar to the one shown in FIG. 9. As shown in FIG. 10, the patient's head can be rested on the second head positioner 92, thereby the patient's eye being positioned in front of an objective lens 98 of the optical assembly such that the eye (pigmented ocular tissue) can be treated with a laser beam produced by any of the three lasers of the third subsystem. In the embodiment of FIG. 10 the laser beam produced by any of the system's laser is delivered to the optical assembly via an optical fiber 99.

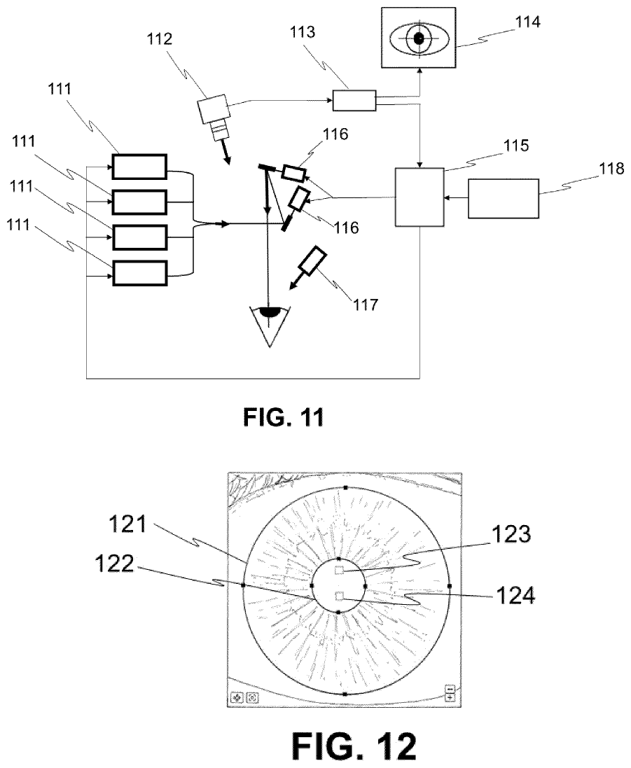

The configuration and operation of the third subsystem of each of the preferred embodiments of FIGS. 9 and 10 is further described with the aid of FIG. 11. As shown in FIG. 11, the third subsystem further comprises control means 115, four lasers 111 of respective different wavelengths, an eye tracker which comprises an electronic unit 113 and a camera 112 which is optically coupled to or integrated with the optical assembly for capturing a live video of the eye and providing the video to the electronic unit 113. Referring to FIG. 11, the electronic unit 113 of the eye tracker is communicatively connected to the control means 115 and is configured to track movements of the eye during the laser scan, and to provide to the control means 115 tracking information related to the tracked movements. A live video 114 of the imaged eye can be displayed on a screen, e.g. on the aforementioned display 95 (computer monitor) shown in FIG. 9. It is noted that the eye tracker and in particular the aforementioned electronic unit 113 may be integrated with or connected to a computer of the third subsystem, or may be replaced by an image/video processing computer program which is configured to track movements of the eye, generate related tracking information during the laser scan and provide said tracking information to the control means 115. Similarly, the control means 115 depicted in FIG. 11 may be an electronic controller or card connectable to a computer, or may be integrated in a computer of the third subsystem. Alternatively or complementary the control means may comprise or be a software program/module which when executed on a computer which has appropriate interfaces for being operationally connected with the lasers and the optical assembly, can perform the functions of the control means. Referring to the preferred embodiment of FIG. 11, the control means 115 is configured to receive and process the set of instructions and the tracking information, to control the optical assembly according to the tracking information and the set of instructions, to select, among the four lasers 111 and according to the set of instructions, the laser that produces the laser beam for each one of the different regions during the laser scan, and to set the values of the laser parameters during the laser scan across the different regions. For this purpose, the control means 115 shown in FIG. 11 is connected and configured to control the lasers 111 and a galvo scanner 116 which comprises two rotatable galvo mirrors. As indicated by the thick arrows in FIG. 11, any of the four lasers 111 of the third subsystem of the shown embodiment may provide a laser beam which, via the galvo scanner 116 and the overall optical assembly of the third subsystem, is directed towards and is scanned across the pigmented ocular tissue of the patient who is appropriately positioned in front of the optical assembly. Referring to FIG. 11, the instructions 118 received by the control means 115 for controlling the lasers 111 and the galvo scanner 116, may comprise the set of instructions, but may also comprise additional or alternative instructions being given by the user of the system or by other hardware or software components of the system during the laser operation. Such additional or alternative instructions may be given/inputted by the user of the system, who is contemplated to be an ophthalmologist or eye surgeon who oversees and supervises the entire procedure, or said additional instruction may originate from optional safety features of the third subsystem, such as for example a digital flaremeter as the one described further below. The third subsystem of the embodiment of FIG. 11 further comprises a LED light 117 which illuminates the eye for imaging the latter. Said LED light is preferably white, but it may be of a different color. When said LED light 117 is white, images captured by the camera 112 can advantageously be used for implementing a digital flaremeter as part of the third subsystem. Said digital flaremeter can be an image processing software which is configured to process images or a video of the eye and detect the presence of any particulates being emitted from the eye or the pigmented ocular tissue during the laser operation. When the presence of such particulates is detected, the digital flaremeter is preferably configured to trigger the control means to stop the laser scan, for safety reasons. Hence, the digital flaremeter may be configured to trigger or provide instructions to the control means to stop the laser scan.

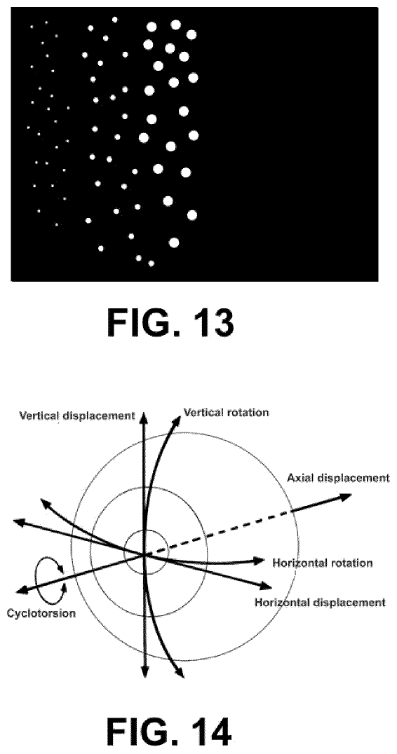

An example of a frame of a video captured by a high-resolution digital camera of an embodiment of the third subsystem is shown in FIG. 12 which further shows two circles 121, 122 which are drawn over the image for indicating the shown boundaries of the pupil and the iris. FIG. 12 further show two small drawn squares 123, 124 over small areas of the pupil. The parts of the images from said small areas of FIG. 12 can be processed for detecting the potential presence thereat of particulates being emitted during the laser treatment. In the digital image/video, the pupil generally has a dark or black color which aids in the detection of said particulates by the aforementioned optional digital flaremeter which is found in a preferred embodiment of the system. Under white light illumination of the eye, within a black/white or grayscale photo taken using a digital camera, such as the camera 112 shown in FIG. 11, said particulates generally may appear as bright or white spots in a black/dark background as shown in FIG. 13. Hence, said particulates can be detected via processing the photo showing said spots. For facilitating this detection, preferably the camera is a high-resolution digital camera. In a preferred embodiment which comprises said digital flaremeter, the latter is configured upon detecting the presence of particulates to trigger an interlock of the control means or directly of the laser, so that the laser is shut down, or the laser beam is blocked, and/or the scanning process stops.

In a preferred embodiment of the disclosure the eye tracker of the third module (i.e. of the third subsystem) of the system according to the disclosure, is a seven-dimensional (7D) eye tracking system which is configured to track the following types of eye movement shown in FIG. 14, each type of movement corresponding to a respective one of the seven dimensions: horizontal displacement (1^{st} dimension); vertical displacement (2^{nd} dimension); horizontal rolling (3^{rd} dimension); vertical rolling (4^{th} dimension); cyclotorsion (5^{th} dimension); axial displacement (6^{th} dimension). The seventh dimension is the time. The use of a 7D eye tracker advantageously improves the safety and positional accuracy of the laser treatment, even at high scanning speeds. By the tracking the eye movement, the eye tracker can provide to the control means information regarding the positional offset of the eye, so that the direction towards which the lase beam is directed by the optical assembly is accordingly corrected (i.e. is offset).

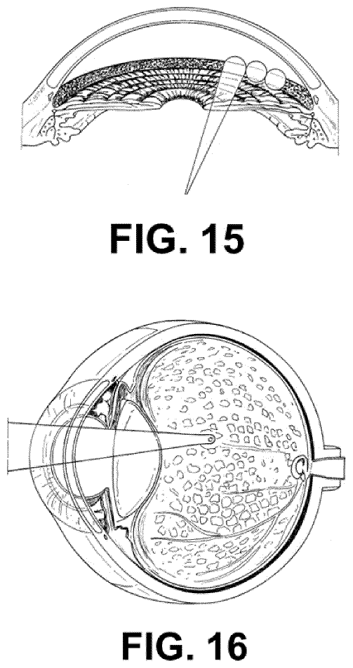

As mentioned, the system according to the disclosure is particularly suitable and generally intended for cosmetic iridoplasty for altering the color of the iris of the eye. The laser treatment applied with the system according to the disclosure may be ablative or non-ablative, depending on the laser parameters used. The system is particularly suitable for enabling the performance of non-ablative laser treatments of melanin containing eye tissues, such as the iris, the trabeculum and the retina. It is contemplated that some of the types of treatment that may be performed with the system of the disclosure are laser trabeculoplasty for glaucoma as shown in FIG. 15, and retinal coagulation by laser for retinal diseases, as shown in FIG. 16. FIG. 15 shows the laser beam being applied on the trabeculum, and for this reason the laser beam being generated with the third subsystem may be directed at an angle towards the trabeculum with the aid of a contact lens being positioned in front of the eye. FIG. 16 shows the laser beam entering through the eye pupil and being applied on the retina of the eye. Most preferably, the first, second and third subsystem of the disclosure are configured to operate in part of fully automatically so that the overall procedure that includes the eye measurements, the planning of the laser procedure, and the laser procedure, is safe, fast and can be completed within few hours, or even within minutes and in less than an hour.

In this text, the term “comprises” and its derivations (such as “comprising”, etc.) should not be understood in an excluding sense, that is, these terms should not be interpreted as excluding the possibility that what is described and defined may include further elements, steps, etc.

In the context of the present disclosure, the term “approximately” and terms of its family (such as “approximate”, etc.) should be understood as indicating values very near to those which accompany the aforementioned term. That is to say, a deviation within reasonable limits from an exact value should be accepted, because a skilled person in the art will understand that such a deviation from the values indicated is inevitable due to measurement inaccuracies, etc. The same applies to the term “about”.

The disclosure is obviously not limited to the specific embodiment(s) described herein, but also encompasses any variations that may be considered by any person skilled in the art (for example, as regards the choice of materials, dimensions, components, configuration, etc.), within the general scope of the disclosure as defined in the claims.

1. A system for a treatment with laser of pigmented ocular tissues, the system comprising a first subsystem (1) for imaging a pigmented ocular tissue of an eye of a person, a second subsystem (2) for planning a laser treatment of the pigmented ocular tissue, and a third subsystem (3) for performing the laser treatment, wherein:
the first subsystem (1) comprises image analysis means (15), a camera, an optical coherence tomography apparatus (12) and at least one first head positioner (13, 14) for positioning a head of the person such that the pigmented ocular tissue can be imaged by the camera and the optical coherence tomography apparatus (12);
the camera is configured to capture at least one color image of the eye and the pigmented ocular tissue;
the optical coherence tomography apparatus (12) is configured to capture at least one optical coherence tomography image of the pigmented ocular tissue;
the image analysis means (15) is configured to process the at least one color image and the at least one optical coherence tomography image for performing a set of measurements which comprise a biometry of the eye, and a densitometry, a pachymetry and a colorimetry of the pigmented ocular tissue, wherein the colorimetry comprises mapping or identifying the color of different regions of the pigmented ocular tissue;
the image analysis means (15) is further configured to produce data related to the set of measurements;
the second subsystem (2) comprises a computer (21) which is configured to process the data related to the set of measurements and to record a set of instructions;
the set of instructions comprises values of laser parameters to be set for a laser scan across different regions of the pigmented ocular tissue, and also comprises an indication of one or more lasers or laser wavelengths to be used during the laser scan for each one of the different regions of the pigmented ocular tissue according to the color of the different regions;
the third subsystem (3) comprises an eye tracker, three or more lasers (111) of respective different wavelengths, an optical assembly (91), control means (115) configured to control the optical assembly (91) and the three or more lasers (111) for executing the laser scan, and a second head positioner (92) for positioning the head of the person such that the pigmented ocular tissue can be treated with a laser beam produced by any of the three or more lasers (111);
the optical assembly (91) is configured to scan the laser beam across the different regions of the pigmented ocular tissue;
the eye tracker is communicatively connected to the control means (115) and is configured to track movements of the eye during the laser scan, and to provide to the control means (115) tracking information related to the tracked movements;
the control means (115) is configured to receive and process the set of instructions and the tracking information, to control the optical assembly (91) according to the tracking information and the set of instructions, to select, among the three or more lasers (111) and according to the set of instructions, the laser that produces the laser beam for each one of the different regions during the laser scan, and to set the values of the laser parameters during the laser scan across the different regions;
the third subsystem (3) further comprises a digital flare meter that is configured to detect the emission of particulates from the eye or the pigmented ocular tissue;
the flare meter upon detecting the emission of particulates during the laser scan is configured to trigger the control means (115) to stop the laser scan.

2. A system according to claim 1, wherein the pigmented ocular tissue comprises the iris, the trabeculum, the retina and/or or any eye tissue that contains melanocytes.

3. A system according to claim 1 or claim 2, wherein the wavelengths of the three or more lasers (111) and/or the recorded values of the laser parameters are such that the laser beam produces an ablation or an apoptosis of melanocytes located on the pigmented ocular tissue during the laser scan, preferably the wavelengths of the three or more lasers (111) and/or the recorded values of the laser parameters being such that the laser beam is non-ablative and produces an apoptosis of the melanocytes during the laser scan.

4. A system according to claim 3, wherein the first subsystem (1), the second subsystem (2) and the third subsystem (3) are integrated with each other forming a single apparatus.

5. A system according to any of the preceding claims, wherein the image analysis means (15) is configured to automatically perform the set of measurements, or any of the biometry, the densitometry, the topography, the pachymetry, and the colorimetry, preferably the colorimetry.

6. A system according to any of the preceding claims, wherein the computer (21) of the second subsystem (2) is configured to generate automatically at least partially the set of instructions, preferably the computer (21) being further configured to enable a manual input or modification by a user of anyone instruction of the set of instructions.

7. A system according to any of the preceding claims, wherein the set of instructions comprises a routine which defines a scanning path to be followed by the laser beam during the laser scan, preferably the computer (21) being configured to automatically generate the routine and/or the control means (115) being configured to control the optical assembly (91) according to the routine, further preferably the scanning path being of a flying-spot scanning path.

8. A system according to any of the preceding claims, wherein:
the laser parameters comprise an energy of the laser beam;
the densitometry comprises mapping or identifying the density of the different regions of the pigmented ocular tissue;
the computer (21) of the second subsystem (2) is configured to record values of the laser beam's energy to be used during the laser scan for each one of the different regions according to the density of the different regions; and preferably the computer (21) of the second subsystem (2) is configured to automatically generate the values of the laser beam's energy as a function of the density of the different regions.

9. A system according to any of the preceding claims, wherein:
the laser parameters comprise a pulse duration of the laser beam;
the pachymetry comprises mapping or identifying the thickness of the different regions of the pigmented ocular tissue;
the computer (21) of the second subsystem (2) is configured to record values of the pulse to be used during the laser scan for each one of the different regions according to the thickness of the different regions; and preferably the computer (21) of the second subsystem (2) is configured to automatically generate the values of the pulse duration as a function of the thickness of the different regions.

10. A system according to any of the preceding claims, wherein the set of measurements comprise a topography of the pigmented ocular tissue, particularly a topography of the different regions of the pigmented ocular tissue.

11. A system according to claim 10, wherein:
the laser parameters comprise a diameter of the laser beam;
the computer (21) of the second subsystem (2) is configured to record values of the diameter to be used during the laser scan for each one of the different regions according to the topography of the different regions; and preferably, the computer (21) of the second subsystem (2) is configured to automatically generate the values of the diameter as a function of the topography of the different regions.

12. A system according to any of the preceding claims, wherein:
the laser parameters comprise a laser pulse frequency;
the biometry comprises identifying one more dimensions of the eye;
the computer (21) of the second subsystem (2) is configured to record values of the laser pulse frequency to be used during the laser scan according to the one or more dimensions of the eye; and preferably, the computer (21) of the second subsystem (2) is configured to automatically generate the values of the laser pulse frequency as a function of the one or more dimensions.

13. A system according to any of the preceding claims, wherein the eye tracker is a seven-dimensional eye tracker.
